(19) United States
(12) Patent Application Publication
(10) Pub. No.: US 2023/0363943 A1
(43) Pub. Date: Nov. 16, 2023

(54) Surgical Instruments For A Trabeculotomy Procedure

(71) Applicant: RAICO International LLC, Westmont, IL (US)

(72) Inventors: Masaki Tanito, Tokyo (JP); Ravi Nallakrishnan, Willowbrook, IL (US)

(21) Appl. No.: 18/028,691

(22) PCT Filed: Oct. 12, 2021
(86) PCT No.: PCT/US2021/054494

§ 371 Date: Mar. 27, 2023

Related U.S. Application Data
(60) Provisional application No. 63/093,382, filed on Oct. 19, 2020.

(51) Int. Cl. A61F 9/007

(52) U.S. Cl. CPC A61F 9/00781; A61B 2217/007

(57) ABSTRACT

A surgical trabeculotomy instrument (40) includes a hand grip portion (42) having an elongated configuration with proximal and distal ends. The instrument (40) includes a first tip portion (48) extending from the distal end of the hand grip portion (42) and further extending along a first tip portion axis (46). A second tip portion (50) extends from the first tip portion (48) along a second tip portion axis (52) and terminates in a sloping face (56) that is transverse relative to the second tip portion axis (52). In one form, at least one of the first tip portion (48) and/or the second tip portion (50) includes a through passage (60) having an inlet (64) for communicating with a fluid supply source and at least one outlet port (68) to permit the flow of an irrigating fluid through the through passage (60) to a target location.

### PRIORITY

This application claims priority of U.S. Provisional Patent Application No. 63/093,382, filed Oct. 19, 2020, the entire contents of which is incorporated herein by reference.

### FIELD OF THE INVENTION

The present invention relates to micro-surgical instruments for performing ophthalmological procedures for treatment of eye diseases, such as glaucoma, and more particularly to surgical instruments having an irrigating tip portion with an inclined distal surface to facilitate a trabeculotomy procedure.

### BACKGROUND OF THE INVENTION

A trabeculotomy is a surgical procedure primarily used to treat congenital glaucoma. It is caused by a developmental arrest of some of the structures within the anterior (front) segment of the eye. These structures include the iris and the ciliary body, which produces the aqueous fluid needed to maintain the integrity of the eye. These structures do not develop normally in the eyes of patients with isolated congenital glaucoma. Instead, they overlap and block the trabecular meshwork, which is the primary drainage system for the aqueous fluid. As a result of this blockage, the trabecular meshwork itself becomes thicker and the drainage holes within the meshwork are narrowed. These changes lead to an excess of fluid in the eye, which can cause pressure that can damage the internal structures of the eye and cause glaucoma.

The purpose of a trabeculotomy is to remove or disrupt a portion of the tissue in the eye to improve the aqueous outflow from the eye, which in turn lowers the intraocular pressure (IOP). Lowering the IOP helps to stabilize the enlargement of the cornea and the distension and stretching of the eye that often occur in congenital glaucoma. The size of the eye, however, will not return to normal. Most importantly, once the aqueous outflow improves, damage to the optic nerve is halted or reversed. The patient's visual acuity may improve after surgery.

The trabeculotomy procedure can restore normal or at least improved drainage of aqueous humor from the eye by removing a portion or segment of the trabecular meshwork, thus allowing the aqueous humor to drain through the open area from which the strip of trabecular meshwork has been opened or removed. The trabeculotomy procedure and certain prior art instruments useable to perform such procedure are described in U.S. Pat. No. 6,979,328, which is hereby incorporated by reference in its entirety. An ab interno trabeculotomy minimally invasive device is described by Tanito, M. Microhook ab interno trabeculotomy, a novel minimally invasive glaucoma surgery. Clinical Ophthalmology. 2017; 12:43-48, which is hereby incorporated by reference in its entirety. The use of an ab interno microhook device for trabeculotomy with cataract surgery is described in by Tanito, M. Effectiveness and safety of combined cataract surgery and microhook ab interno trabeculotomy in Japanese eyes with glaucoma: report of an initial case series. Jpn J Ophthalmol. 2017; 61:457-464, which is hereby incorporated by reference in its entirety. A comparison of devices for trabeculotomy-related surgeries is described by Tanito, M. et al. Ab interno trabeculotomy-related glaucoma surgeries. Taiwan J Ophthalmol. 2019; 9:67-71, which is hereby incorporated by reference in its entirety. Further studies of the ab interno surgical device are described by Iwasaki, K. et al. Clinical practice preferences for glaucoma surgery in a Japan: a survey of Japan Glaucoma Society specialists. Jpn J Ophthalmol. 2020:64:385-391, and by Omoto, T. et al. Comparison of the short-term effectiveness and safety profile of ab interno combined trabeculotomy using 2 types of trabecular hooks. Jpn J Ophthalmol. 2020:64:407-413, which are both hereby incorporated by reference in their entireties as if fully set forth herein.

At present there remains a need in the art for the development of improved, easy to use, inexpensive, minimally invasive instruments useable to perform the trabeculotomy procedure, or other similar procedures, to reduce intraocular pressure.

### BRIEF SUMMARY OF THE INVENTION

In accordance with one broad form of the present invention, a micro-surgical instrument or hook is disclosed which is particularly configured to facilitate performing trabeculotomy such as for treatment of glaucoma. The instrument includes a specifically configured tip portion which facilitates disruption of the trabecular meshwork to permit drainage of aqueous humor to enhance the vision of the patient.

In one preferred form of the invention, the instrument includes a hand grip portion having an elongated configuration, with proximal and distal ends. The instrument includes a first tip portion extending from the distal end of the hand grip portion and defines a first tip portion axis. The instrument includes a second tip portion extending from the first tip portion that terminates in a sloping face that is transverse relative to a second tip portion axis. At least one of the first tip portion and/or the second tip portion includes a through passage having an inlet for communicating with an irrigating fluid supply source and at least one outlet port to permit the flow of an irrigating fluid through the through passage to a target location.

In one preferred form of the invention, the sloping face of the instrument second tip portion is an ellipse.

In yet another form of the invention, the second tip portion and the first tip portion are connected by a bend. In one preferred form of the invention, the second tip portion axis is angled relative to the first tip portion axis by about 90 degrees.

According to one preferred form of the invention, the at least one outlet port comprises a pair of oppositely facing outlet ports located in the first tip portion proximate to a bend between the first tip portion and the second tip portion. In one preferred form, the oppositely facing outlet ports are located in the first tip portion to direct flow of an irrigating fluid in directions normal to each one of: (i) the first tip portion axis; and (2) the second tip portion axis.

According to another preferred form of the invention, the at least one outlet port comprises: a first outlet port located in the first tip portion proximate to a bend between the first tip portion and the second tip portion; and a second outlet port located in the second tip portion. In a preferred form of the invention, the first outlet port is located on the first tip portion to direct the flow of an irrigating fluid in a direction parallel to the second tip portion axis, and the second outlet port is located in the second tip portion to direct the flow of an irrigating fluid in a direction parallel to the first tip portion axis.

In still another form of the present invention, the at least one outlet port is located in the sloping face of the second tip portion to direct the flow of an irrigating fluid in a direction parallel to the second tip portion axis.

In another form of the present invention, the at least one outlet port is located in a bend between the first tip portion and the second tip portion to direct the flow of an irrigating fluid in a direction parallel to the second tip portion axis.

In one form of the present invention, the at least one outlet port is located in a bend between the first tip portion and the second tip portion to direct the flow of an irrigating fluid in a direction parallel to the first tip portion axis.

According to another preferred form of the invention, the at least one outlet port is located in the second tip portion to direct the flow of an irrigating fluid in a direction that is transverse to the second tip portion axis.

In yet another preferred form of the invention, wherein the sloping face is substantially flat and defines an arcuate leading edge and an arcuate trailing edge. The leading edge extends axially beyond the trailing edge, relative to the second tip portion axis.

In still another form of the invention, the second tip portion has a length of about 0.6 mm, as measured along the second tip portion axis from the first tip portion axis to the distalmost or leading edge of the second tip portion.

In one preferred form of the present invention, the sloping face is angled about 50 degrees relative to the second tip portion axis.

In another form of the present invention, the hand grip portion includes either: (i) a reservoir for accommodating an irrigating fluid; or (ii) a connection for communicating with an external irrigation fluid supply source.

According to one form of the present invention, an intermediate tip portion extends between the first tip portion and the second tip portion, wherein the intermediate tip portion is connected to the first tip portion by a first bend and the intermediate tip portion is connected to the second tip portion by a second bend such that the second tip portion and the intermediate tip portion have a V-shaped configuration when viewed in a plane containing the first tip portion axis and the second tip portion axis.

According to another form of the present invention, an intermediate tip portion extends between the first tip portion and the second tip portion, wherein the intermediate tip portion is connected to the first tip portion by a first bend and the intermediate tip portion is connected to the second tip portion by a second bend such that the second tip portion and the intermediate tip portion have a U-shaped configuration when viewed in a plane containing the first tip portion axis and the second tip portion axis.

In still another form of the present invention, the sloping face is rectangular. In a preferred form, the first tip portion has a substantially square cross-sectional shape in a plane that extends in a normal direction relative to the first tip portion axis, and further the second tip portion has a substantially square cross-sectional shape in a plane that extends in a normal direction relative to the second tip portion axis. In this preferred form, the first tip portion axis and the second tip portion axis generally reside within a shared plane, and define a pair of opposite planar surfaces.

According to another form of the present invention, at least the first tip portion and the second tip portion of the instrument are formed from a substrate with at least one coating deposited thereon. Preferably, the substrate is silicon and the at least one coating deposited thereon is carbon.

In accordance with one broad form of the present invention, a method of using a surgical instrument to treat the trabecular meshwork of the eye is disclosed. The method includes a first step of obtaining an inventive surgical instrument having at least a hand grip portion having an elongated configuration, with proximal and distal ends. The instrument includes a first tip portion extending from the distal end of the hand grip portion and defines a first tip portion axis. The instrument includes a second tip portion extending from the first tip portion that terminates in a sloping face that is transverse relative to a second tip portion axis. At least one of the first tip portion and/or the second tip portion includes a through passage having an inlet for communicating with an irrigating fluid supply source and at least one outlet port to permit the flow of an irrigating fluid through the through passage to a target location. The method includes the further step of gripping the handle portion to bring the sloping face into contact with the trabecular meshwork. The method includes the further step of introducing an irrigating fluid into the surgical instrument to direct flow of the irrigating fluid from the at least one outlet port to the trabecular meshwork.

In accordance with another broad form of the invention, the instrument includes a hand grip portion having an elongated configuration, with proximal and distal ends. The instrument includes a first tip portion extending from the distal end of the hand grip portion and defines a first tip portion axis. The instrument includes a second tip portion extending from the first tip portion that terminates in a sloping face that is transverse relative to a second tip portion axis. The first tip portion axis and the second tip portion axis generally reside within a shared plane, and define a pair of opposite planar surfaces located on either side of the first and second tip axes.

### BRIEF DESCRIPTION OF THE DRAWINGS

In the accompanying drawings forming part of the specification, in which like numerals are employed to designate like parts throughout the same,

FIG. 1 is a perspective view, taken from below, of a first embodiment of a surgical instrument according to the present invention;

FIG. 1A is a right side elevation view of the instrument of FIG. 1 configured as an ab interno left hook;

FIG. 1B is a greatly enlarged, fragmentary, right side elevation view of the circled portion of the instrument shown in FIG. 1A;

FIG. 1C is an enlarged, front elevation view, of the instrument of FIG. 1;

FIG. 1D is a greatly enlarged, fragmentary, front elevation view of the circled portion of the instrument shown in FIG. 1C;

FIG. 2 is a perspective view, taken from above, of another embodiment of a surgical instrument according to the present invention configured as an ab interno right hook;

FIG. 2A is a left side elevation view of the instrument of FIG. 2;

FIG. 2B is a greatly enlarged, fragmentary, left side elevation view of the circled portion of the instrument shown in FIG. 2A;

FIG. 2C is an enlarged, front elevation view, of the instrument of FIG. 2;

FIG. 2D is a greatly enlarged, fragmentary, front elevation view of the circled portion of the instrument shown in FIG. 2C;

FIG. 3 is a fragmentary, top plan view of an operative, distal end of another embodiment of a surgical instrument according to the present invention configured as a straight hook;

FIG. 3A is a left side elevation view of the instrument of FIG. 3;

FIG. 3B is a greatly enlarged, fragmentary, left side elevation view of the circled portion of the instrument shown in FIG. 3A;

FIG. 3C is an enlarged, perspective view, taken from above, of the instrument of FIG. 3;

FIG. 3D is a greatly enlarged, fragmentary, perspective view, taken from above, of the distal end portion of the instrument shown in FIG. 3C;

FIG. 3E is a greatly enlarged, fragmentary, perspective view, taken from above and from the rear, of the distal end portion of the instrument shown in FIG. 3C;

FIG. 4 is a greatly enlarged, fragmentary, perspective view, taken from above, of the distal end portion of another embodiment of an instrument according to the present invention, and FIG. 4 shows the instrument having a pair of oppositely facing lateral irrigating ports with irrigation fluid flow shown by black arrows;

FIG. 5 is a greatly enlarged, fragmentary, perspective view, taken from above, of the distal end portion of still another embodiment of an instrument according to the present invention, and FIG. 5 shows the instrument having a pair of irrigating ports arranged in an orthogonal manner with irrigation fluid flow shown by black arrows;

FIG. 6 is a greatly enlarged, fragmentary, perspective view, taken from above, of the distal end portion of another embodiment of an instrument according to the present invention, and FIG. 6 shows the instrument having a single top irrigating port with irrigation fluid flow shown by black arrows;

FIG. 7 is a greatly enlarged, fragmentary, perspective view, taken from above, of the distal end portion of another embodiment of an instrument according to the present invention, and FIG. 7 shows the instrument having a single bottom irrigating port with irrigation fluid flow shown by black arrows;

FIG. 8 is a greatly enlarged, fragmentary, perspective view, taken from above, of the distal end portion of another embodiment of an instrument according to the present invention, and FIG. 8 shows the instrument having a single front irrigating port with irrigation fluid flow shown by black arrows;

FIG. 9 is a greatly enlarged, fragmentary, perspective view, taken from above, of the distal end portion of another embodiment of an instrument according to the present invention, and FIG. 9 shows the instrument having a single front and top irrigating port with irrigation fluid flow shown by black arrows;

FIG. 10 is an enlarged, fragmentary, right side elevation view of another embodiment of a surgical instrument according to the present invention, and FIG. 10 shows only a distal, operative portion of the instrument having a single irrigation port in the top of the instrument;

FIG. 11 is an enlarged, fragmentary, left side elevation view of another embodiment of a surgical instrument according to the present invention, and FIG. 11 shows only a distal, operative portion of the instrument having a single irrigation port in the top of the instrument;

FIG. 12 is a diagrammatic, simplified view of the instrument of the present invention in communication with a fluid supply source;

FIG. 13 is a greatly enlarged, fragmentary, perspective view, taken from above, of another embodiment of a surgical instrument according to the present invention, and FIG. 13 shows only a distal, operative portion of the instrument;

FIG. 14 is an enlarged, fragmentary, left side view, of the instrument shown in FIG. 13; and

FIG. 15 shows a cross-sectional view of the instrument of FIG. 14, taken along view line 15-15 in FIG. 14.

### DETAILED DESCRIPTION OF THE PREFERRED EMBODIMENTS

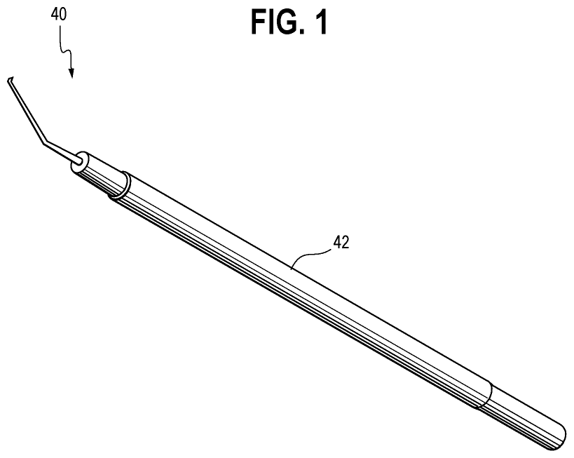

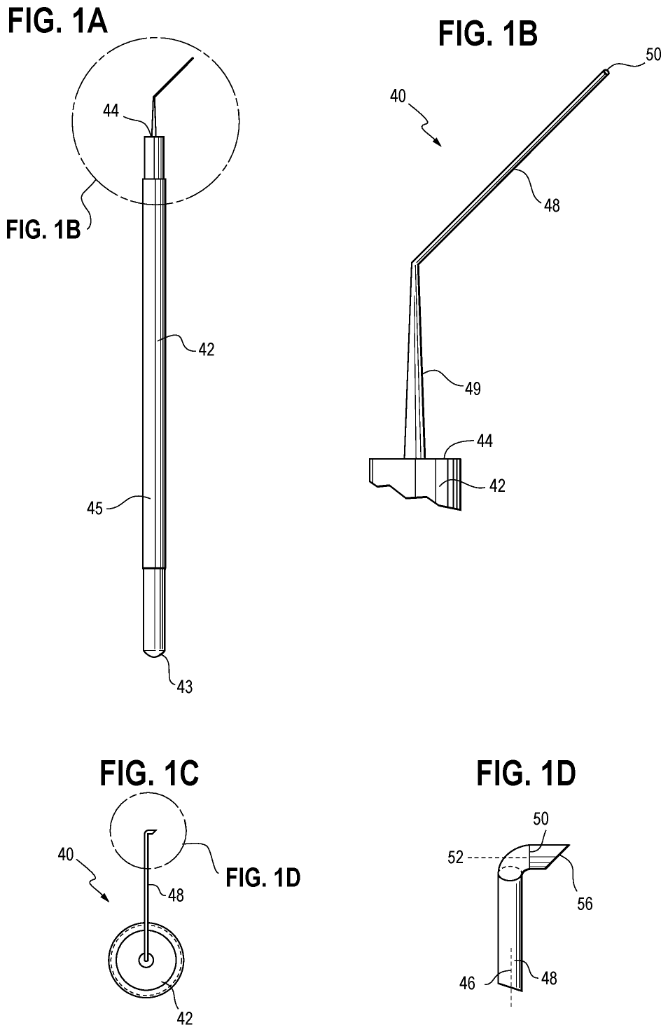

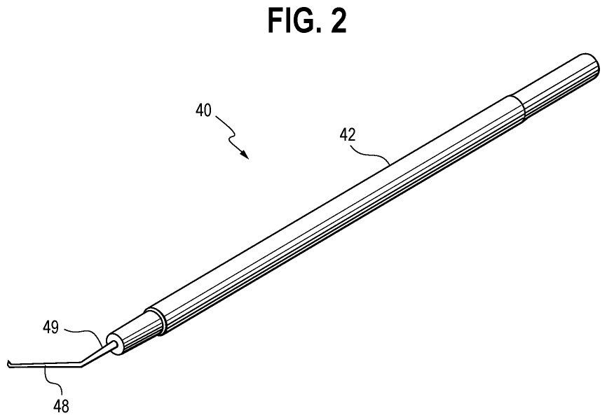

With reference to FIGS. 1-1D, in accordance with first illustrated embodiment of the present invention, a present trabeculotomy surgical instrument 40 includes a hand piece or hand grip portion 42 for being gripped by a user of the instrument 40 and having an elongated configuration, including a proximal end 43 and a distal end 44. FIGS. 1-1D show a first variation of the instrument 40 for use as an ab interno left-angled instrument. FIGS. 2-2D show an ab interno right-angled variation of the instrument. FIGS. 3-3E show a straight variation of the instrument. Each of these variations of the instrument 40 are configured to contact specific angles, arcs, or portions of the trabecular meshwork of both of a patient's eyes, as will be discussed in detail below. The hand grip portion 42 can be provided with either a rounded or a flattened configuration and is preferably knurled for ease of grip. Furthermore, the hand grip portion 42 may be a cannula hub or fitting with means for being attached to a larger machine, irrigation system, or commercial irrigating handpiece (e.g., screw threading, snap-fit connection, luer lock connection, friction fit, locked, etc.). The numbered features of the embodiments of the instrument 40 illustrated and discussed herein are designated generally with number where such features are analogous in structure and function.

With reference now to FIG. 1B, the instrument 40 includes a first tip portion 48 that extends, either directly from the distal end 44 of the hand grip portion 42, or indirectly from the distal end 44 of the hand grip portion 42 via one or more straight or angled shank portions 49 depending on the right, left, or straight designed use of the instrument 40.

As can be seen in FIG. 1D, the first tip portion 48 defines a first tip portion axis 46 through its geometric center. A second tip portion 50 extends from the first tip portion 48 and defines a second tip portion axis 52 through the geometric center of the second tip portion 50. The second tip portion 50 terminates in a sloping face 56 that is dimensioned to perform the desired tearing, scraping, and/or disruption of the trabecular meshwork. The second tip portion 50 is joined to the first tip portion 48 by a bend 58 (FIG. 3B).

In one presently preferred form of the instrument 40, the bend 58 is a right-angle bend such that the second tip portion 50 and its central axis 52 are about 90 degrees relative to the first tip portion 48 and its central axis 46.

The first tip portion 48 and the second tip portion 50 in the embodiments of the present invention illustrated in FIGS. 1-3E are generally tubular and have a circular cross-sectional shape for the majority of their lengths, when viewed in a plane that is normal to their central axes 46, 52. Preferably, the first tip portion 48 and the second tip portion 50 have a diameter of between about 0.2 mm to about 0.3 mm, and more preferably a diameter of about 0.22 mm.

With reference to FIG. 3B, the operative, sloping face or surface 56 of the instrument 40 has the form of an ellipse and is angled or transverse relative to the axis 52 of the second tip portion 50 by an angle of between about 40 degrees and about 60 degrees, and more preferably by an angle of about 50 degrees. The sloping face 56 has an arcuate leading edge 72 and an arcuate trailing edge 76. The distalmost point of the leading edge 72 is about 0.6 mm from the central axis 46 of the first tip portion 48.

The sloping face 56 in the embodiments illustrated in FIGS. 1-3B is preferably solid, and the sloping face 56 may be formed by cutting the tubular end of the second tip portion 50. The first and second tip portions 48 and 50 may be formed or made from metal, a molded plastic material, or it may be insert-molded from two different materials to form a composite. In one form, the instrument tip portions 48 and 50 may be stamped from a round wire to achieve its final shape.

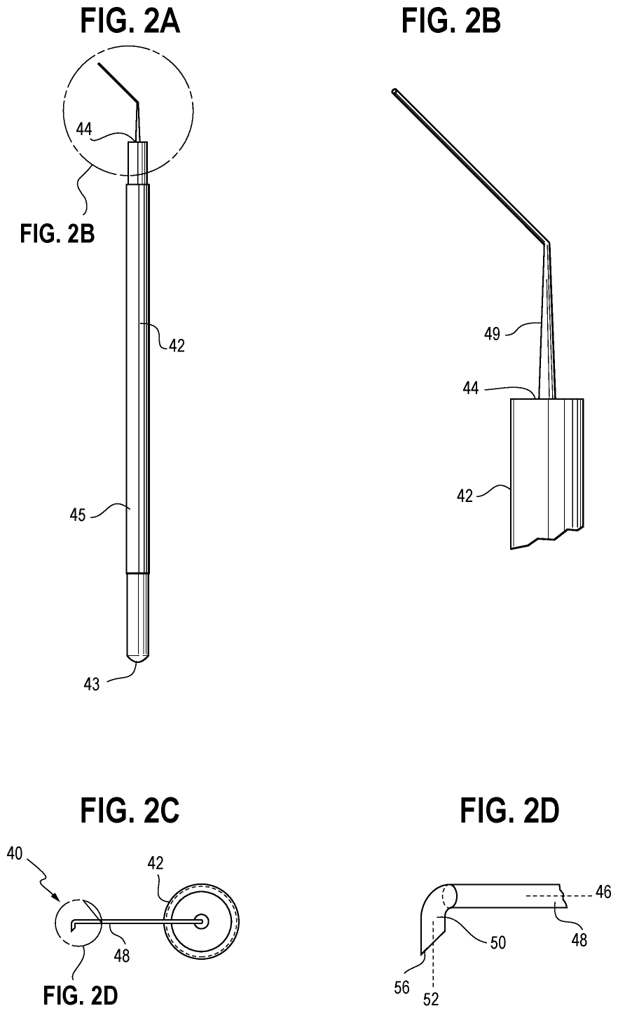

Referring next to FIG. 4, another embodiment of the instrument 40 is illustrated having a through passage 60 that communicates via an inlet 64 with an irrigation fluid supply source (either located in a reservoir 45 in the hand grip portion 42, or located in an external pressurized container or machine and connected to the hand grip portion 42 through tubing). The through passage 60 extends from the inlet 64 in the first tip portion 48 and at least through the first tip portion 48. The through passage 60 may further extend through the second tip portion 50 in some embodiments, which will be discussed in detail below. The through passage 60 terminates in one or more outlet ports 68 to permit flow of an irrigating fluid through the instrument 40 to a target location proximate to the trabecular meshwork. The hand grip portion 42, or other machinery or system connected to the hand grip portion 42, includes a pressure switch, collapsible wall, button, bellows, etc. or conventional or non-conventional means for actuating the instrument 40 to cause an on-demand or selective flow of irrigating fluid to be expelled from the outlet port or ports 68 to a target location. There are many commercially available irrigating handpieces or systems on the market, and it will be understood that the instrument 40 may be adapted to function with such handpieces or systems.

In the embodiment of the instrument 40 illustrated in FIG. 4, the through passage 60 terminates in a pair of oppositely facing, lateral outlet ports 68 to direct flow in directions that are normal to both the first tip portion axis 46 and the second tip portion axis 52 (i.e., out of the plane shared by both axes 46 and 52). In this embodiment, the ports 68 are preferably located proximate to the bend 58. The sloping face 56 is solid for engaging or disrupting the trabecular meshwork without removing any strips of tissue. The flow of irrigating fluid is shown generally as black line arrows from the inlet 64 to the outlet ports 68 in directions to assist in irrigating areas around the trabecular meshwork. The lateral directions of the flow illustrated in FIG. 4, extending orthogonally into and out of the plane containing the axes 46 and 52, may be particularly suited for maintaining the anterior chamber depth during surgery.

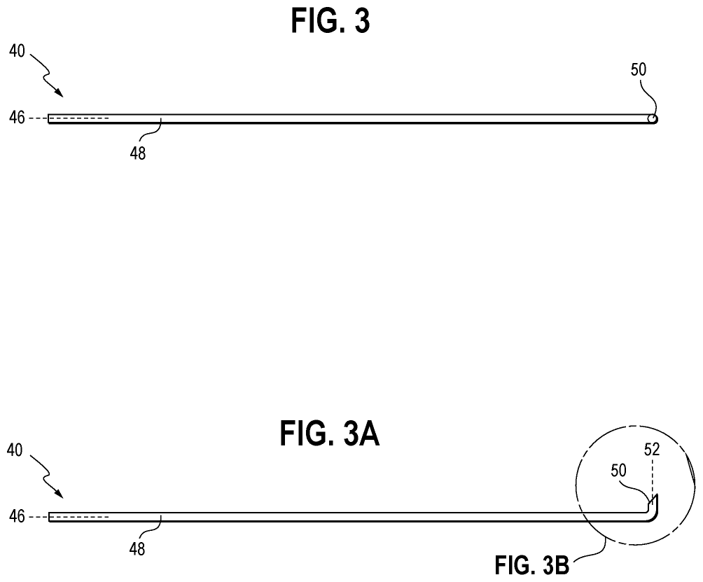

In another embodiment of the instrument 40, illustrated in FIG. 5, the through passage 60 terminates in a first outlet port 68 in the first tip portion 48 to direct flow in a direction that is parallel to the second tip axis 52 (downwardly pointing arrow in FIG. 5). The passage 60 further terminates in a second outlet port 68 in the second tip portion 50 to direct flow in a direction that is generally parallel to the first tip axis 48 (right-pointing arrow in FIG. 5). In this embodiment, the sloping face 56 is solid for engaging the trabecular meshwork without removing tissue. The flow of irrigating fluid is shown as arrows from the inlet 64 to the outlet ports 68 in directions to assist in irrigating the trabecular meshwork in case of blood or viscoelastic clean up. The directions of the flow illustrated in FIG. 5 may be particularly suited for the following: (i) opening of the post Schlemm's Canal lumen, such as the collector channels; (ii) securing visibility during surgery; and/or (iii) maintaining the anterior chamber depth during surgery.

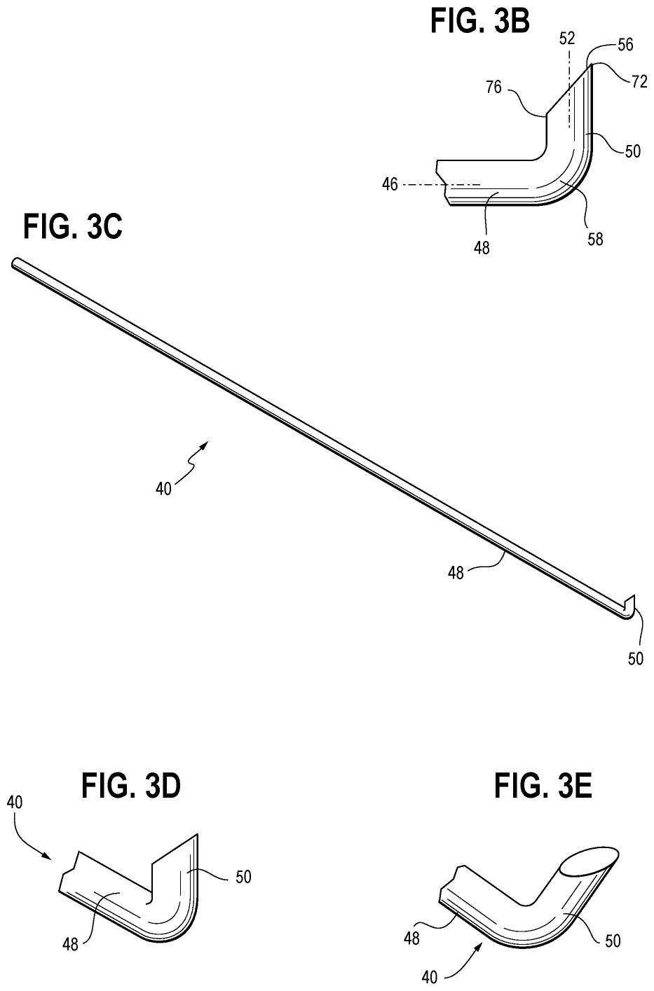

In another embodiment of the instrument 40, illustrated in FIG. 6, the through passage 60 terminates in a single outlet port 68 in the second tip portion 50 to direct flow in a direction that is parallel to the second tip axis 52 (upward-pointing arrow in FIG. 6). In this embodiment, the sloping face 56 contains the outlet port 68 for engaging the trabecular meshwork. The flow of irrigating fluid is shown as arrows from the inlet 64 to the unitary outlet port 68 in a direction to assist in irrigating the trabecular meshwork in case of blood or viscoelastic clean up. The direction of the flow illustrated in FIG. 6 may be particularly suited for the following: (i) maintaining the Schlemm's Canal lumen space during goniotomy; (ii) opening of the post Schlemm's Canal lumen, such as the collector channels; and/or (iii) securing visibility during surgery.

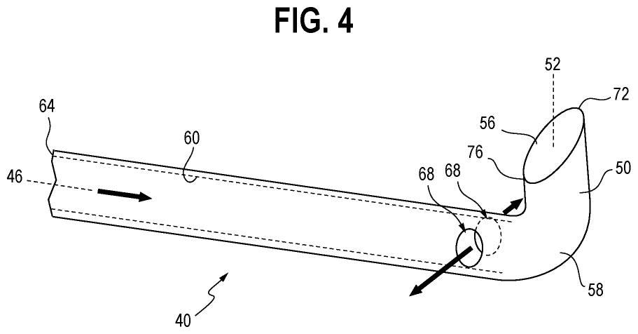

In an embodiment of the instrument 40, illustrated in FIG. 7, the through passage 60 terminates in a single outlet port 68 in the second tip portion 50 to direct flow in a direction that is parallel to the second tip axis 52 (downwardly pointing arrow in FIG. 7). In this embodiment, the sloping face 56 is solid for engaging the trabecular meshwork. The flow of irrigating fluid is shown as arrows from the inlet 64 to the unitary outlet port 68 in a direction to assist in irrigating areas below the trabecular meshwork. The direction of the flow illustrated in FIG. 7 may be particularly suited for the following: (i) maintaining the anterior chamber depth during surgery; and/or (ii) securing visibility during surgery.

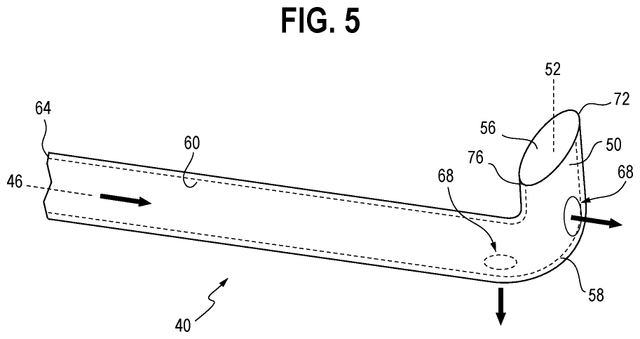

In the embodiment of the instrument 40 illustrated in FIG. 8, the through passage 60 terminates in a single outlet port 68 that is located in the bend 58 connecting the first and second tip portions 48, 50 to direct flow in a direction that is generally parallel to the first tip axis 46 (right-pointing arrow in FIG. 8). In this embodiment, the sloping face 56 is solid for engaging the trabecular meshwork. The flow of irrigating fluid is shown as arrows from the inlet 64 to the unitary outlet port 68 in a direction to assist in irrigating the trabecular meshwork in case of blood or viscoelastic clean up. The direction of the flow illustrated in FIG. 8 may be particularly suited for opening of the post Schlemm's Canal lumen, such as the collector channels.

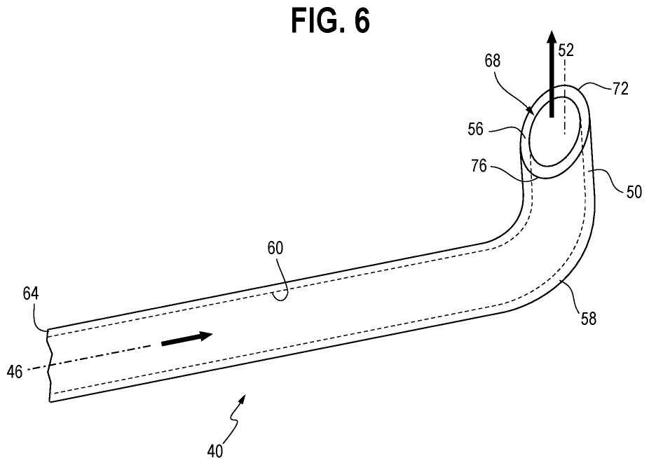

In the embodiment of the instrument 40 illustrated in FIG. 9, the through passage 60 terminates in a single outlet port 68 that is located in the second tip portion 50, at an intermediate location between the bend 58 and the leading edge 72 of the sloping face 56, to direct flow in a direction that is transverse or angled between about 20 degrees and about 45 degrees relative to the second tip axis 52 (upper-right pointing arrow in FIG. 9). In this embodiment, the sloping face 56 is solid for engaging the trabecular meshwork. The flow of irrigating fluid is shown as arrows from the inlet 64 to the unitary outlet port 68 in a direction to assist in irrigating the trabecular meshwork in case of blood or viscoelastic clean up. The direction of the flow illustrated in FIG. 9 may be particularly suited for opening of the post Schlemm's Canal lumen, such as the collector channels.

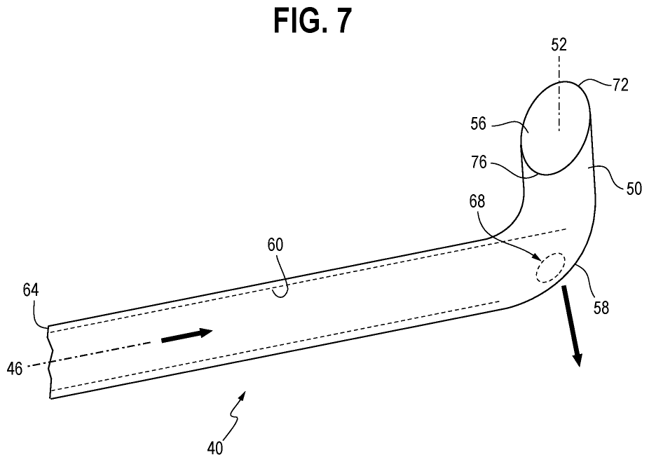

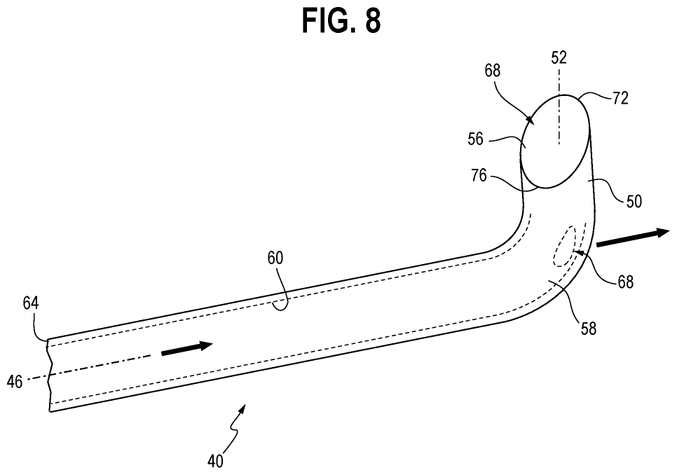

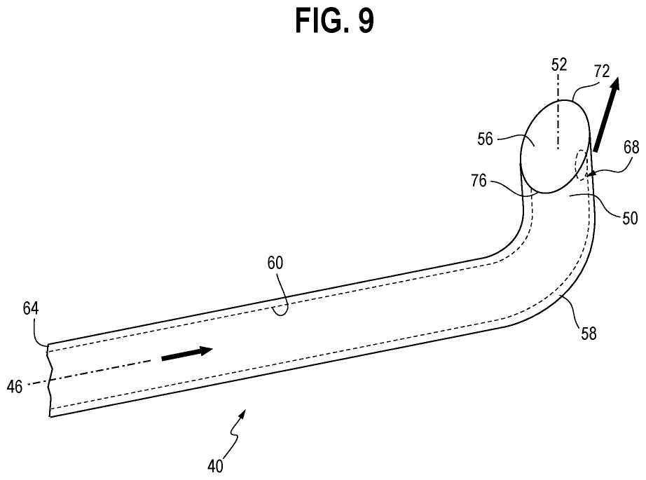

In some forms of the instrument 40 and with reference to FIGS. 10 and 11, at least the first and second tip portions 48, 50 are separated by an intermediate tip portion 80 extending therebetween. The intermediate tip portion 80 is connected to the first tip portion 48 by a first bend 84, and the intermediate tip portion 80 is connected to the second tip portion 50 by a second bend 88.

With reference to FIG. 10, the instrument distal or operative tip portions may have a V-shaped or reflex bend configuration when viewed normal to a plane containing the first tip portion axis 46 and the second tip portion axis 52 (as illustrated).

With reference to FIG. 11, the instrument distal or operative tip portions may have a U-shaped or reflex bend configuration when viewed in a plane containing the first tip portion axis 46 and the second tip portion axis 52 (as illustrated).

The instruments 40 disclosed in FIGS. 10 and 11 may be more suitable for engaging the trabecular meshwork for some procedures.

The illustrated embodiments of the instruments 40 in FIGS. 4-11 may be alternatively formed without an irrigation flow passage 60 or ports 68 for use in a non-irrigating manner for reduced cost of manufacture.

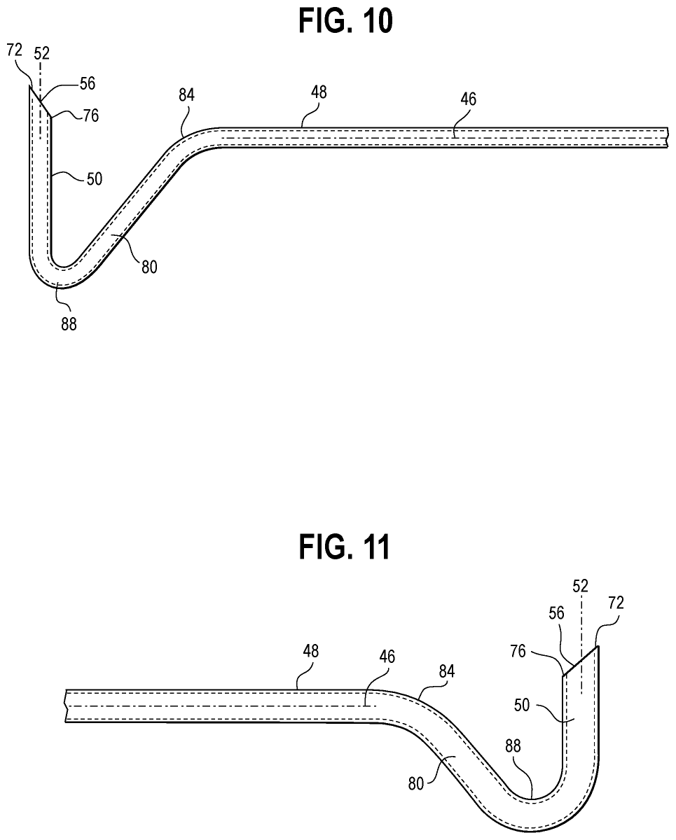

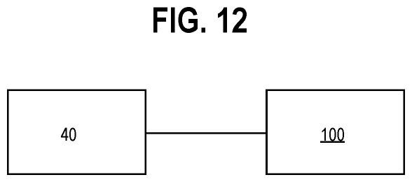

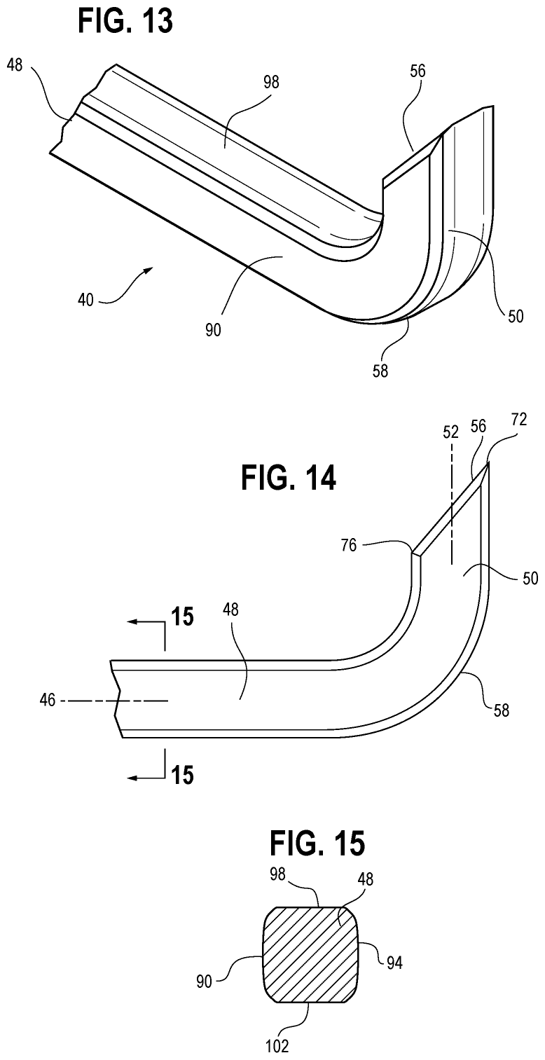

With reference now to FIGS. 13-15, another embodiment of the instrument 40 is illustrated, which has a somewhat planar configuration. The instrument 40 includes the same features of a tip first portion 48 extending along a first axis 46 and a tip second portion 50 extending along a second axis 52. With reference to FIG. 15, it can be seen that the cross-section of the tip portions 48 and 52, which are substantially uniform, have the form of a rounded rectangle, defining a first pair of opposite, lateral sides 90, 94 and a pair of opposite, top and bottom sides 98, 102. The sloping face 56 further has a generally rounded rectangular configuration that extends between a leading edge 72 and a trailing edge 76. It will be understood that the instrument 40 illustrated in FIGS. 13-15 may have any type of hand grip portion 42, and any number of straight or angled shank portions 49, depending on the right, left, or straight designed use of the instrument 40.

As can be seen in FIG. 1D, the first tip portion 48 defines a first tip portion axis 46 through its geometric center. A second tip portion 50 extends from the first tip portion 48 and defines a second tip portion axis 52 through the geometric center of the second tip portion 50. The second tip portion 50 terminates in a sloping face 56 that is dimensioned to perform the desired tearing, scraping, and/or disruption of the trabecular meshwork. The second tip portion 50 is joined to the first tip portion 48 by a bend 58.

In one presently preferred form of the instrument 40, the first tip portion 48 and the second tip portion 50 have a nominal width and thickness of between about 0.2 mm to about 0.3 mm, and the operative, sloping face 56 of the instrument 40 is angled or transverse relative to the axis 52 of the second tip portion 50 by an angle between about 40 degrees and about 60 degrees, and more preferably about 60 degrees. The distalmost point of the leading edge 72 is about 0.7 mm from the bottom surface 102 of the first tip portion 48.

The sloping face 56 in the embodiment illustrated in FIGS. 13-15 is preferably solid, and the tip portions 48, 50 of the instrument 40 are preferably formed by chemical vapor deposition of carbon on a silicon wafer substrate for improved manufacturing tolerances and manufacturing costs. The first and second tip portions 48 and 50 may alternatively be formed or made from metal, a molded plastic material, or it may be insert-molded from two different materials to form a composite. In one form, the instrument tip portions 48 and 50 may be stamped or cut from a plate or wire to achieve its final shape.

The inventors believe that the embodiments of the instrument 40 discussed above may advantageously permit the surgeon to disrupt the trabecular meshwork by trabeculotomy and reducing the post-operative Schlemm's canal resistance (i.e., opening of collector channels like canaloplasty) by irrigating flow. The instrument 40 may lower costs for the trabeculotomy procedure compared to existing surgical instruments with expensive handpieces, and may improve surgical outcomes by reducing the time of the procedure compared to surgeries performed with prior art devices. The instruments 40 advantageously work inside the meshwork without the need to remove it, such as is done in some prior art devices, and further the size of the instruments 40 are significantly smaller than some prior art devices. The instruments 40 are preferably on the order of about 200 microns, and the Schlemm's Canal is about 200-250 microns when dilated. Further, the dull leading edge and sloping end face 56 of the instrument provides an improved efficacy compared to traditional goniotomy that is performed with a sharp knife. Such prior art devices can further increase the risk of damage to the outer wall of the canal. The instruments 40 may be easier to use by a surgeon, cost less than leading prior art devices on the market, and/or reduce or eliminate post-operative visits to the surgeon by the patient.

The inventors have further found that the instruments 40 as described above are especially suitable for eyes with shallow anterior chamber, such as phakic eye, primary angle-closure glaucoma (PACG), older subjects, child glaucoma, and patients of Asian descent. The operative tip portions of the instruments 40, which have a relatively smaller size compared to prior art devices such as the Kahook dual blade having a large distal tip portion, are less prone to damage of tissues other than the trabecular meshwork (such as the Descemet membrane and iris root). The instruments 40 enable a more selective relief of the trabecular meshwork resistance compared to the Kahook dual blade. In addition, the instruments 40 described herein, when provided in a set of three variations of a right-angled instrument, a left-angled instrument, and straight instrument, the set enables an incision or disruption of the trabecular meshwork of an angle of about 240 degrees, which is significantly wider than the Kahook dual blade angle of about 90 degrees. Furthermore, the instruments 40 may be manufactured and sold at a significantly lower cost than the Kahook dual blade in both reusable and disposable forms.

It will be understood that the instrument 40 may be formed in a variety of sizes for small incision glaucoma surgery or regular glaucoma surgery. Furthermore, the various embodiments of the instrument 40, having different configurations of outlet port or ports 68, may be color coded in the tip portions or hand grip portion 42 to assist the user in differentiating between the different configurations of the instrument 40.

In one presently preferred method of use of the instrument 40 may be configured in a straight hook instrument (e.g., FIGS. 3-3E) for use in incising the nasal angle of the right eye and/or the left eye. The straight hook is inserted through a temporal incision in the cornea of the operative eye. About a 120-degree arc of the nasal angle of the trabecular meshwork is then engaged by the sloping face 56 of the instrument. The sloping face 56 is sized and shaped to enter the Schlemm's canal, and the trabecular meshwork is incised by circumferential movement of the instrument 40 along the 120-degree arc. A gonioprism may be used to view the trabecular meshwork as it is engaged by the instrument 40. Irrigation fluid can be applied on-demand by the user of the instrument 40 in a reflux burst or jet pulse of about 5-8 cc of fluid to the target location through the passage 60 to the one or more outlet ports 68.

The instrument 40 may further be configured in a right-angled hook instrument (e.g., FIGS. 2-2D) and left-angled hook instrument (e.g., FIGS. 1-1D) for use in incising the temporal angle of the right eye and/or left eye. The right-angled hook and left-angled hook configuration are inserted through a nasal position incision in the cornea of the operative eye. About a 120-degree arc of the temporal angle of the trabecular meshwork is then engaged by the sloping face 56 of the instruments 40. The sloping face 56 is sized and shaped to enter the Schlemm's canal, and the trabecular meshwork is incised by circumferential movement of the instrument 40 along the 120-degree arc. A gonioprism may be used to view the trabecular meshwork engaged by the instrument 40. Irrigation fluid may be applied on-demand by the user of the instrument 40 in reflux bursts or jet pulses to the target location through one or more outlet ports 68 in 5-8 cc pulses of fluid applied.

Various modifications and alterations to this invention will become apparent to those skilled in the art without departing from the scope and spirit of this invention. Illustrative embodiments and examples are provided as examples only and are not intended to limit the broadest scope of the present invention.

1. A trabeculotomy surgical instrument, comprising:
a hand grip portion having an elongated configuration, having proximal end and a distal end;
a first tip portion extending from the distal end of said hand grip portion and extending along a first tip portion axis; and
a second tip portion extending from said first tip portion along a second tip portion axis, said second tip portion terminating in a sloping face that is transverse relative to said second tip portion axis (52), at least one of said first tip portion and/or said second tip portion includes a through passage having an inlet for communicating with an irrigating fluid supply source and at least one outlet port to permit the flow of an irrigating fluid through said through passage to a target location.

2. The trabeculotomy surgical instrument in accordance with claim 1, wherein said sloping face is an ellipse.

3. The trabeculotomy surgical instrument in accordance with claim 1, wherein said second tip portion and said first tip portion are connected by a bend.

4. The trabeculotomy surgical instrument in accordance with claim 3, wherein said second tip portion axis is angled about 90 degrees relative to said first tip portion axis.

5. The trabeculotomy surgical instrument in accordance with claim 1, wherein said at least one outlet port comprises a pair of oppositely facing outlet ports located in said first tip portion proximate to a bend between said first tip portion and said second tip portion.

6. The trabeculotomy surgical instrument in accordance with claim 5, wherein said pair of oppositely facing outlet ports are located in said first tip portion to direct flow of an irrigating fluid in directions normal to each said first tip portion axis; and said second tip portion axis.

7. The trabeculotomy surgical instrument in accordance with claim 1, wherein said at least one outlet port comprises: a first outlet port located in said first tip portion proximate to a bend between said first tip portion and said second tip portion; and a second outlet port located in said second tip portion.

8. The trabeculotomy surgical instrument in accordance with claim 7, wherein said first outlet port is located on said first tip portion to direct flow of an irrigating fluid in a direction parallel to said second tip portion axis, and said second outlet port is located in said second tip portion to direct flow of an irrigating fluid in a direction parallel to said first tip portion axis.

9. The trabeculotomy surgical instrument in accordance with claim 1, wherein said at least one outlet port is located in said sloping face of said second tip portion to direct flow of an irrigating fluid in a direction parallel to said second tip portion axis.

10. The trabeculotomy surgical instrument in accordance with claim 1, wherein said at least one outlet port is located in a bend between said first tip portion and said second tip portion to direct flow of an irrigating fluid in a direction parallel to said second tip portion axis.

11. The trabeculotomy surgical instrument in accordance with claim 1, wherein said at least one outlet port is located in a bend between said first tip portion and said second tip portion to direct flow of an irrigating fluid in a direction parallel to said first tip portion axis.

12. The trabeculotomy surgical instrument in accordance with claim 1, wherein said at least one outlet port is located in said second tip portion to direct flow of an irrigating fluid in a direction that is transverse to said second tip portion axis.

13. The trabeculotomy surgical instrument in accordance with claim 1, wherein said sloping face is substantially flat and defines an arcuate leading edge and an arcuate trailing edge, said leading edge extending axially beyond said trailing edge, relative to said second tip portion axis.

14. The trabeculotomy surgical instrument in accordance with claim 1 further comprising an intermediate tip portion extending between said first tip portion and said second tip portion, wherein said intermediate tip portion is connected to said first tip portion by a first bend and said intermediate tip portion is connected to said second tip portion by a second bend such that said second tip portion and said intermediate tip portion have a V-shaped configuration when viewed in a plane containing said first tip portion axis and said second tip portion axis.

15. The trabeculotomy surgical instrument in accordance with claim 1 further comprising an intermediate tip portion extending between said first tip portion and said second tip portion, wherein said intermediate tip portion is connected to said first tip portion by a first bend and said intermediate tip portion is connected to said second tip portion by a second bend such that said second tip portion and said intermediate tip portion have a U-shaped configuration when viewed in a plane containing said first tip portion axis and said second tip portion axis.

16. The trabeculotomy surgical instrument in accordance with claim 1, wherein said second tip portion has a length of about 0.6 mm along said second tip portion axis.

17. The trabeculotomy surgical instrument in accordance with claim 1, wherein said sloping face is angled about 50 degrees relative to said second tip portion axis.

18. The trabeculotomy surgical instrument in accordance with claim 1, wherein said hand grip portion includes a reservoir for accommodating an irrigating fluid stored therein.

19. The trabeculotomy surgical instrument in accordance with claim 1, wherein said hand grip portion includes a connection for accommodating a flow of an irrigating fluid accommodating from an external irrigating fluid supply source.

20. The trabeculotomy surgical instrument in accordance with claim 1, wherein said sloping face is rectangular.

21. The trabeculotomy surgical instrument in accordance with claim 1 wherein said first tip portion has a substantially square cross-sectional shape in a plane that extends in a normal direction relative to said first tip portion axis, and said second tip portion has a substantially square cross-sectional shape in a plane that extends in a normal direction relative to said second tip portion axis.

22. The trabeculotomy surgical instrument in accordance with claim 1 wherein said first tip portion axis and said second tip portion axis generally reside within a shared plane, and define a pair of opposite planar surfaces.

23. The trabeculotomy surgical instrument in accordance with claim 1 wherein at least said first tip portion and said second tip portion are formed from a substrate with at least one coating deposited thereon.

24. The trabeculotomy surgical instrument in accordance with claim 23 wherein said substrate is silicon and said at least one coating deposited thereon is carbon.

25. A method of using a trabeculotomy surgical instrument to treat the trabecular meshwork of the eye, the method comprising the steps of:
obtaining the trabeculotomy surgical instrument of claim 1;
gripping the hand grip portion to bring one of said sloping face into contact with the trabecular meshwork of an eye; and
introducing an irrigating fluid into said surgical instrument to direct flow of the irrigating fluid from said at least one outlet port to the trabecular meshwork.

26. A trabeculotomy surgical instrument, comprising:
a hand grip portion having an elongated configuration, having proximal end and a distal end;
a first tip portion extending from the distal end of said hand grip portion and extending along a first tip portion axis; and
a second tip portion extending from said first tip portion along a second tip portion axis, said second tip portion terminating in a sloping face that is transverse relative to said second tip portion axis, wherein said first tip portion axis and said second tip portion axis generally reside within a shared plane, and define a pair of opposite planar surfaces.
